(19) United States
(12) Patent Application Publication
(10) Pub. No.: US 2006/0105333 A1
(43) Pub. Date: May 18, 2006

(54) Method for diagnosis of intestinal-type gastric tumors

(75) Inventors: Yusuke Nakamura, Yokohama-shi (JP); Yoichi Furukawa, Kawasaki-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kawasaki-shi (JP); The University of Tokyo, Bunkyo-ku (JP)

(21) Appl. No.: 10/520,881

(22) PCT Filed: Jul. 8, 2003
(86) PCT No.: PCT/JP03/08651

§ 371 Date: Oct. 17, 2005

Related U.S. Application Data
(60) Provisional application No. 60/394,941, filed on Jul. 10, 2002.

(51) Int. Cl. C12Q 1/68; G01N 33/574; A61K 48/00; A61K 39/00

(52) U.S. Cl. 435/6; 435/7.23; 514/44; 424/185.1

(57) ABSTRACT

Objective methods for detecting and diagnosing intestinal-type gastric cancers are described herein. Also described is method for predicting the presence or absence of lymph node metastasis (i.e., identifying the metastatic phenotype). In one embodiment, the diagnostic method involves the scoring of gene expression profiles that discriminate between lymph node positive tumors and lymph node negative tumors. The predictive score calculated acts as diagnostic indicator that can objectively indicate whether a sample tissue has the metastatic phenotype. The present invention further provides methods of diagnosing intestinal-type gastric cancer in a subject, methods of screening for therapeutic agents useful in the treatment of intestinal-type gastric cancer, methods of treating intestinal-type gastric cancer and method of vaccinating a subject against intestinal-type gastric cancer.

### PRIORITY INFORMATION

This application claims priority to U.S. Provisional Application Ser. No. 60/394,941, filed Jul. 10, 2002.

### TECHNICAL FIELD

The present invention relates to the field of cancer research. More particularly, the present invention relates to the detection of intestinal-type gastric tumors. The invention further relates to methods of diagnosing intestinal-type gastric tumors in a subject, methods of screening for therapeutic agents useful in the treatment of intestinal-type gastric tumors, methods of treating intestinal-type gastric tumors and method of vaccinating a subject against intestinal-type gastric tumors.

### BACKGROUND OF THE INVENTION

The invention relates to detection and diagnosis of tumors, particularly intestinal-type gastric tumors.

Gastric cancer is a leading cause of cancer death in the world, particularly in the Far East, with approximately 700,000 new cases diagnosed worldwide annually. Surgery is the mainstay in terms of treatment, because chemotherapy remains unsatisfactory. Gastric cancers at an early stage can be cured by surgical resection, but prognosis of advanced gastric cancers remains very poor.

The vast majority (90-95%) of gastric cancers are gland-forming adenocarcinomas. Other less common tumors of the stomach include lymphomas, carcinoids and gastric stromal tumors. Epidemiologic studies have shown that the two major histologic subtypes of gastric adenocarcinomas—the intestinal (well differentiated) type and diffuse (poorly differentiated) type—arise by distinct pathways. The intestinal type is strongly associated with Helicobacter pylori, and usually arises on a backdrop of chronic gastritis, gastric atrophy, and intestinal metaplasia. In contrast, poorly differentiated adenocarcinomas are usually not associated with these changes. Clinically, the latter often present with diffuse thickening of the stomach wall, rather than a discernible mass (linitis plastica). The intestinal adenocarcinomas have a better prognosis than the diffuse variant, most of which have metastasized and spread beyond the confines of the stomach at the time of diagnosis.

As with other cancers, stage is the most important determinant of outcome. A factor in determining the prognosis of solid tumors in humans is lymph node metastasis, an independent risk factor for recurrence of gastric cancer. Although the expression of some genes has been associated with lymph node metastasis, the molecular mechanisms involved remain unclear.

The present invention represents a marked improvement in the field of intestinal-type gastric cancer detection and diagnosis. Prior to the invention, knowledge of genes involved in intestinal-type gastric cancer was fragmentary. The information described herein provides genome-wide information about how gene expression profiles are altered during multi-step carcinogenesis and metastasis. Specifically, the present invention describes “marker” genes that are either up-regulated or down-regulated in intestinal type gastric tumors as compared to non-tumor tissues. The information disclosed herein not only contributes to a more profound understanding of gastric cancer tumorigenesis and metastasis, particularly of the intestinal-type, but also provide indicators for developing novel strategies to diagnose, treat, and ultimately prevent intestinal-type gastric cancer.

### SUMMARY OF THE INVENTION

Accordingly, the present invention provides diagnostic methods that correlate the expression of marker genes to the presence or absence of intestinal-type gastric cancer. More particularly, the present invention provides sensitive, specific and convenient diagnostic methods for distinguishing between benign and malignant lesions and for identifying the presence or absence of lymph-node metastasis (i.e., identifying the metastatic phenotype).

The invention is based on a genome-wide analysis of gene expression analysis using laser-capture microdissection techniques and cDNA microarrays. The analysis led to a definition of “marker genes”, i.e., genes that are over-expressed (up-regulated) or under-expressed (down-regulated) in intestinal-type gastric cancers. These genes represent new therapeutic targets and biomarkers for this disease. Gene expression patterns, which correlate with a metastatic phenotype were also defined. The invention therefore provides a sensitive, specific and convenient diagnostic and prognostic method for gastric cancers.

Also within the invention is a method of determining whether a tumor is metastatic by comparing the level of expression of a gene in the tumor compared to a control value. The gene is selected from the list provided in FIG. 2, preferably DDOST, GNS, NEDD8, LOC51096, CCT5, CCT3, PPP2R1B and two ESTs (GENBANK™ Accession Nos. AA533633 and AI755112) genes can be used as up-regulated gene. An increase in the level of expression in the tumor compared to the control value indicates that the tumor is metastatic. Alternatively, the method is carried out by comparing the level of expression of a gene in the tumor compared to a control value in which the gene is selected from the genes listed in FIG. 2, preferably UBQLNI1, AIM2, and USP9X genes can be used as down-regulated gene. A decrease in the level of expression in the tumor compared to the control value indicates that the tumor is metastatic.

A method of screening for a therapeutic agent useful in treating or preventing intestinal-type gastric cancer is provided. The method includes contacting a candidate compound with a cell expressing marker genes listed in Table 1 and Table 2, and selecting a compound that reduces the expression level of the up-regulated marker genes shown in Table 1 or enhances the expression of the down-regulated marker genes shown in Table 2.

The present invention further provides a method of screening for a therapeutic agent useful in treating intestinal-type gastric cancer, wherein the method includes administering a candidate compound to a test animal, and measuring the expression level of the marker genes, and selecting a compound that reduces or enhances the expression level of the marker genes.

The present invention further provides a method of screening for a therapeutic agent useful in treating intestinal-type gastric cancer, wherein the method includes contacting a candidate compound with a cell into which a vector comprising the transcriptional regulatory region of the marker genes and a reporter gene has been introduced, and measuring the activity of said reporter gene, and selecting a compound that reduces the expression level of said reporter gene.

Furthermore, the present invention provide a method of screening for a therapeutic agent useful in treating intestinal-type gastric cancer, wherein the method includes contacting a candidate compound with a protein encoded by a marker gene, and measuring the activity of said protein; and selecting a compound that reduces the activity of said protein.

Other features and advantages of the invention will be apparent from the following detailed description and from the claims. However, it is to be understood that both the foregoing summary of the invention and the following detailed description are of a preferred embodiment, and not restrictive of the invention or other alternate embodiments of the invention.

### BRIEF DESCRIPTION OF THE DRAWINGS

FIG. 1 is a dot plot showing a validation of microarray data by quantitative RT-PCR. The scatter-plot shows the logarithmic expression ratio (Cy3/Cy5) of each sample obtained by the array (left) and by quantitative RT-PCR (right).

FIG. 2A is a diagram showing genes whose expression differed between node-positive (N+) and node-negative (N−) tumor classes. The logarithmic expression ratio of each sample is shown. The right column contains discriminant coefficients calculated by forward stepwise discriminant function analysis. Forward stepwise discriminant function analysis identified five genes (shown in bold type) as independent “predictors”.

FIG. 2B is a dot plot showing the results of discriminant function analysis. The scatter-plot shows the “predictive” (discriminant) scores for the node-positive (N+) and node-negative (N−) classes. Group centroids are denoted by horizontal bars.

### DETAILED DESCRIPTION

In the context of the present invention, the following definitions apply:

The present invention relates to the diagnosis and treatment of gastric cancers of the intestinal type, which is also known as intestinal adenocarcinoma.

Tumors of the intestine and gastric epithelium are classified as benign, malignant or pre-malignant. In the context of the present invention, the term “intestinal tumors” encompasses benign, malignant and pre-malignant tumors of the epithelium of the stomach and intestine. The term “intestinal-type gastric cancer” refers to a malignant state, characterized by uncontrolled, abnormal growth of cells. Cancer cells can spread locally or through the blood stream and lymphatic system to other parts of the body.

A “carcinoma” is a malignant new growth of cells that arises from the epithelium. Carcinomas are cancerous tumors that tend to infiltrate into adjacent tissue and metastasize to distant organs. An adenocarcinoma is a specific type of carcinoma arising from the lining of the walls of an organ, such as the stomach or intestine. Herein, the terms “carcinoma” and “adenocarcinoma” are used interchangeably. There is a clear need in the art for new methods for diagnosing, treating and preventing intestinal adenocarcinoma, particularly at the early stages—before to the carcinoma metastasizes to other organ systems.

An “adenoma” is a benign epithelial tumor in which the cells form a recognizable glandular structure or in which the cells are clearly derived from glandular epithelium. Intestinal-type gastric cancers are believed to develop through the “adenoma-to-carcinoma sequence” model in the literature. Accordingly, in gastric tumors, adenoma is the pre-malignant phase of gastric carcinoma. Early detection and diagnosis of adenoma is useful in preventing the onset of carcinoma. Likewise, the treatment and prevention of adenoma can protect the progressing into intestinal-type gastric carcinoma in a subject.

In the context of the present invention, the term “metastatic” refers to the spread of a disease from the organ or tissue of origin to another part of the body.

The present invention describes genes that discriminate between intestinal tumors and non-cancerous mucosae as well as genes that discriminate between metastatic intestinal-type gastric cancer and non-metastatic intestinal-type gastric cancer. Such genes are herein collectively referred to as “marker genes”. The present invention demonstrates that the expression of such marker genes can be analyzed to distinguish between malignant and benign tumors of the intestine and metastatic intestinal-type gastric cancer (e.g., lymph node positive tumors) from non-metastatic intestinal-type gastric cancer (e.g., lymph node negative tumors).

The term “expression profile” as used herein refers to a collection of expression levels of a number of genes. In the context of the present invention, the expression profile preferably comprises marker genes that discriminate between metastatic and non-metastatic gastric cancer. The present invention involves the step of analyzing expression profiles of marker genes to determine if a sample displays characteristics of intestinal-type gastric cancer, thereby distinguishing metastatic cancers from non-metastatic cancers and diagnosing the presence of intestinal-type gastric cancer in a subject.

The term “characteristics of a intestinal-type gastric cancer” is used herein to refer to a pattern of alterations in the expression levels of a set of marker genes which is characteristic to intestinal-type gastric cancer. Specifically, certain marker genes are described herein either up-regulated (i.e., those of Table 1) or down-regulated (i.e., those of Table 2) in intestinal-type gastric cancer. When the expression level of one or more up-regulated marker genes included in the expression profile is elevated as compared with that in a control, the expression profile can be assessed as having the characteristics of intestinal-type gastric cancer. Likewise, when the expression level of one or more down-regulated marker genes included in the expression profile is lowered as compared with that of a control, the expression profile can be assessed as having the characteristics of intestinal-type gastric cancer. When, not all, but most of the pattern of alteration in the expression levels constituting the expression profile is characteristic to intestinal-type gastric cancer, the expression profile is assessed to have the characteristics of intestinal-type gastric cancer.

In the context of the present invention, expression profiles can be obtained by using a “DNA array”. A “DNA array” is a device that is convenient for comparing expression levels of a number of genes at the same time. DNA array-based expression profiling can be carried out, for example, by the method as disclosed in “Microarray Biochip Technology” (Mark Schena, Eaton Publishing, 2000), etc.

A DNA array comprises immobilized high-density probes to detect a number of genes. In the present invention, any type of polynucleotide can be used as probes for the DNA array. Preferably, cDNAs, PCR products, and oligonucleotides are useful as probes. Thus, expression levels of many genes can be estimated at the same time by a single-round analysis. Namely, the expression profile of a specimen can be determined with a DNA array. The DNA array-based method of the present invention comprises the following steps of:
(1) synthesizing aRNAs or cDNAs including those of marker genes;
(2) hybridizing the aRNAs or cDNAs with probes for the marker genes; and
(3) detecting the aRNA or cDNA hybridizing with the probes and quantifying the amount of mRNA thereof.

The term “aRNA” refers to RNA transcribed from a template cDNA with RNA polymerase (amplified RNA). An aRNA transcription kit for DNA array-based expression profiling is commercially available. With such a kit, aRNA can be synthesized using T7 promoter-attached cDNA as a template with 17 RNA polymerase. Alternatively, by PCR using random primer, cDNA can be amplified using, as a template, a cDNA synthesized from mRNA.

The DNA array may further comprise probes, which have been spotted thereon, to detect the marker genes of the present invention. There is no limitation on the number of marker genes spotted on the DNA array. For example, one may select 5% or more, preferably 20% or more, more preferably 50% or more, still more preferably 70% or more of the marker genes of the present invention. Genes other than the marker genes may be also spotted on the DNA array. For example, a probe for a gene whose expression level is not significantly altered may be spotted on the DNA array. Such a gene can be used for normalizing assay results to compare assay results of multiple arrays or different assays.

A “probe” is designed for each selected marker gene, and spotted on a DNA array. Such a “probe” may be, for example, an oligonucleotide comprising 5-50 nucleotide residues. A method for synthesizing such oligonucleotides on a DNA array is known to those skilled in the art. Longer DNAs can be synthesized by PCR or chemically. A method for spotting long DNA, which is synthesized by PCR or the like, onto a glass slide is also known to those skilled in the art. A DNA array that is obtained by the method as described above can be used for diagnosing intestinal-type gastric cancer according to the present invention.

The prepared DNA array is contacted with aRNA, followed by the detection of hybridization between the probe and aRNA. The aRNA can be previously labeled with a fluorescent dye. A fluorescent dye such as Cy3(red) and Cy5 (green) can be used to label an aRNA. aRNA s from subject and control are labeled with different fluorescent dyes, respectively. The difference in the expression level between the two can be estimated based on a difference in the signal intensity. The signal of fluorescent dye on the DNA array can be detected by a scanner and analyzed using a special program. For example, the Suite from Affymetrix is a software package for DNA array analysis.

The compound isolated by the screening is a candidate for drugs that inhibit the activity of the protein encoded by marker genes and can be applied to the treatment or prevention of intestinal adenocarcinoma.

Moreover, compound in which a part of the structure of the compound inhibiting the activity of proteins encoded by marker genes is converted by addition, deletion and/or replacement are also included in the compounds obtainable by the screening method of the present invention.

When administrating the compound isolated by the method of the invention as a pharmaceutical for humans and other mammals, such as mice, rats, guinea-pigs, rabbits, chicken, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80™ and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and an anti-oxidant. The prepared injection may be filled into a suitable ampule.

Methods well known to one skilled in the art may be used to administer the pharmaceutical composition of the present invention to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kg of body-weight.

As noted above, antisense nucleic acids corresponding to the nucleotide sequence of a marker gene can be used to reduce the expression level of the marker gene. Antisense nucleic acids corresponding to marker genes that are up-regulated in intestinal-type gastric carcinoma are useful for the treatment of intestinal-type gastric carcinoma. Specifically, the antisense nucleic acids of the present invention may act by binding to the marker genes or mRNAs corresponding thereto, thereby inhibiting the transcription or translation of the genes, promoting the degradation of the mRNAs, and/or inhibiting the expression of proteins encoded by the marker genes, finally inhibiting the function of the proteins. The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

The antisense nucleic acid derivatives of the present invention act on cells producing the proteins encoded by marker genes by binding to the DNAs or mRNAs encoding the proteins, inhibiting their transcription or translation, promoting the degradation of the mRNAs, and inhibiting the expression of the proteins, thereby resulting in the inhibition of the protein function.

Also, a siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which RNA is transcribed. In the context of the present invention, the siRNA comprises a sense nucleic acid sequence and an anti-sense nucleic acid sequence against an up-regulated marker gene, such as those set forth in Table 1. The siRNA is constructed such that a single transcript has both the sense and complementary antisense sequences from the target gene, e.g., a hairpin.

The method is used to alter the expression in a cell of an up-regulated, e.g., as a result of malignant transformation of the cells. Binding of the siRNA to a transcript corresponding to one of the up-regulated marker genes of Table 1 in the target cell results in a reduction in the protein production by the cell. The length of the oligonucleotide is at least 10 nucleotides and may be as long as the naturally-occurring the transcript. Preferably, the oligonucleotide is 19-25 nucleotides in length. Most preferably, the oligonucleotide is less than 75, 50, 25 nucleotides in length.

The nucleotide sequence of the siRNAs was designed using a siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene to evaluate.

The antisense oligonucleotide or siRNA of the invention inhibit the expression of the polypeptide of the invention and is thereby useful for suppressing the biological activity of the polypeptide of the invention. Also, expression-inhibitors, comprising the antisense oligonucleotide or siRNA of the invention, are useful in the point that they can inhibit the biological activity of the polypeptide of the invention. Therefore, a composition comprising the antisense oligonucleotide or siRNA of the present invention is useful in treating a cell proliferative disease such as cancer.

An antisense nucleic acid or siRNA derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids or siRNA derivative is given to the patient by directly applying onto the ailing site or by injecting into a blood vessel so that it will reach the site of ailment. An antisense-mounting medium can also be used to increase durability and membrane-permeability. Examples are, liposomes, poly-L-lysine, lipids, cholesterol, lipofectin or derivatives of these.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of present invention include modified oligonucleotides. For example, thiolated nucleotides may be used to confer nuclease resistance to an oligonucleotide.

The present invention further provides a method of determining whether a tumor is metastatic, comprising comparing the level of expression of a gene in said tumor compared to a control value, wherein said gene is selected from the group consisting of DDOST, GNS, NEDD8, LOC51096, CCT5, CCT3, PPP2R1B and two ESTs (GENBANK™ Accession Nos. AA533633 and AI755112) and wherein an increase in the level of expression in said tumor compared to said control value indicates that the tumor is metastatic.

Alternatively, the present invention provides a method of determining whether a tumor is metastatic, comprising comparing the level of expression of a gene in said tumor compared to a control value, wherein said gene is selected from the group consisting of UBQLN1, AIM2, and USP9X and wherein a decrease in the level of expression in said tumor compared to said control value indicates that the tumor is metastatic.

In another embodiment, the present invention provides a method for diagnosing intestinal-type gastric cancer in a subject comprising the steps of:
(a) detecting an expression level of one or more marker genes in a specimen collected from a subject to be diagnosed, wherein the one or more marker genes is selected from the group consisting of the genes listed in Table 1 and the genes listed in Table 2; and
(b) comparing the expression level of the one or more marker genes to that of a control, wherein high expression level of a marker gene from Table 1 or a low expression level of a marker gene from Table 2, as compared to control, is indicative of intestinal-type gastric cancer.

In additionally, the present invention provides a method of predicting lymph node-negative cancers and/or lymph node-positive cancers, the method comprising the steps of:
(a) detecting an expression level of one or more marker genes in a specimen collected from a subject to be predicted, wherein the one or more marker genes is selected from the group consisting of DDOST, GNS, NEDD8, LOC51096, CCT5, CCT3, PPP2R1B, two ESTs (GENBANK Accession Nos. AA533633 and AI755112), UBQLN1, AIM2, and USP9X; and
(b) comparing the expression level of the one or more marker genes to that of a control, wherein a high expression level or low expression level of a marker gene selected from the group consisting of DDOST, GNS, NEDD8, LOC51096, CCT5, CCT3, PPP2R1B, and two ESTs (GENBANK Accession Nos. AA533633 and AI755112), as compared to the control, is indicative of lymph node-positive cancers or lymph node-negative cancers, respectively, or wherein a low expression level or high expression level of a marker gene selected from the group consisting of UBQLN1, AIM2, and USP9X, as compared to the control, is indicative of lymph node-positive cancers or lymph node-negative cancers.

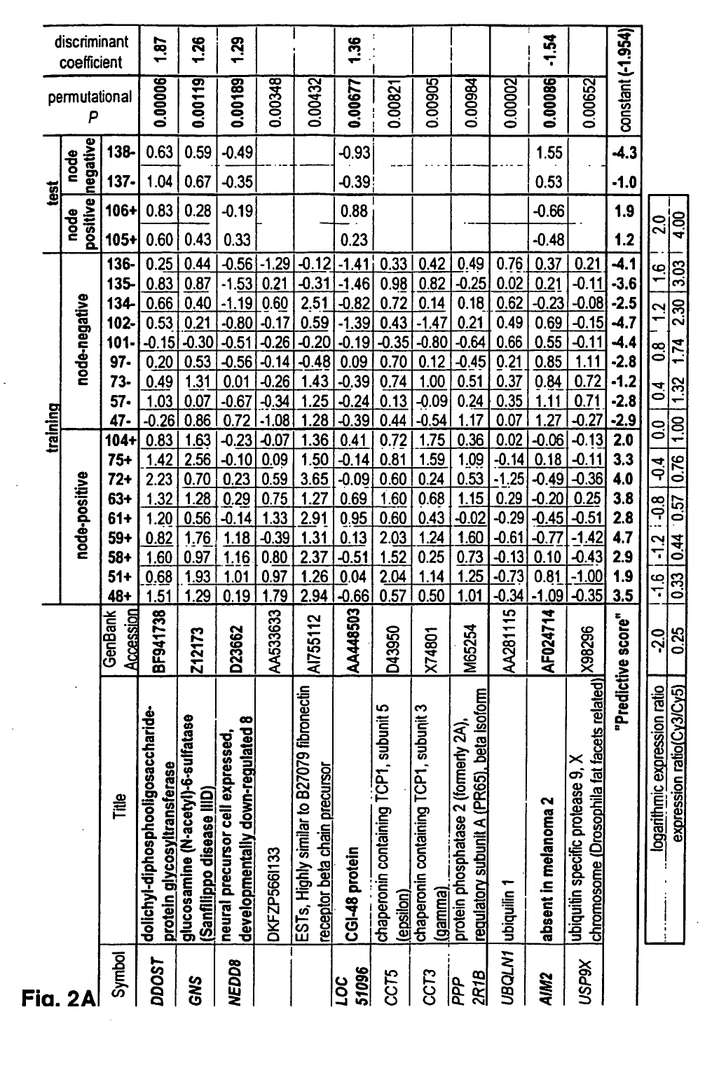

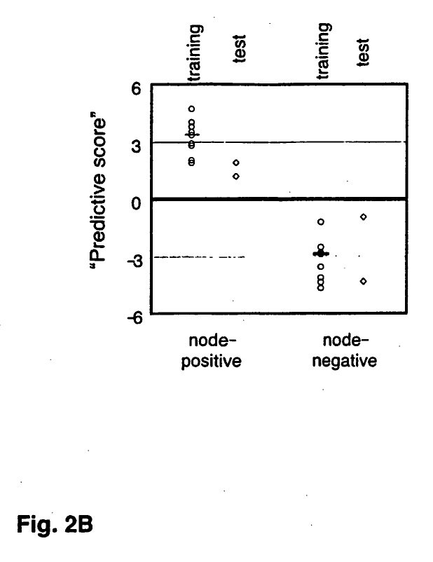

In the present invention, marker gene(s) may be at least one gene selected from the group consisting of DDOST, GNS, NEDD8, LOC51096, CCT5, CCT3, PPP2R1B, two ESTs (GENBANK Accession Nos. AA533633 and AI755112), UBQLN1, AIM2, and USP9X (FIG. 2a). Among them, preferably, DDOST, GNS, NEDD8, LOC51096, and AIM2 may be selected as marker genes. In the present invention, the 5 genes have been named “predictor”. More preferably, the expression level of all of DDOST, GNS, NEDD8, LOC51096, and AIM2 can be detected. Then, the expression level of the marker gene(s) can be compared to normal control.

In an alternate embodiment, the method of the present invention involves the step of scoring expression profiles for genes that discriminate between lymph node-negative cancers and/or lymph node-positive cancers. The steps of the method include receiving expression profiles for genes selected as differentially expressed in lymph node-negative cancers versus lymph node-positive cancers (i.e., “marker genes”) and determining a function of the log ratios of the expression profiles over the selected genes. The step of “determining a function of the log ratios of the expression profiles over the selected genes” may comprise summing the weighted log ratios of the expression profiles over the selected genes. The weight for each gene is assigned a first value when the average log ratio is higher for lymph node-positive cancers than for lymph node-negative cancers and a second value when the average log ratio is lower for lymph node-positive cancers than for lymph node-negative cancers. Preferably, the second value is substantially the opposite of the first value, e.g., the first value is 1 and the second value is −1. In one embodiment, the method of the present invention involves the scoring of gene expression profiles that discriminate between lymph node positive tumors and lymph node negative tumors. The predictive score calculated acts as diagnostic indicator that can objectively indicate whether a sample tissue has the metastatic phenotype. For example, step (b) in the prediction method may comprise the steps of determining a function of the log ratios of the expression profiles over the selected genes comprising summing the weighted log ratios of the expression profiles over the selected genes, wherein the weight for each gene is a first value when the average log ratio is higher for lymph node-positive cancers than for lymph node-negative cancers and a second value when the average log ratio is lower for lymph node-negative cancers than for lymph node-positive cancers.

In the present invention, a method for predicting lymph node-negative cancers and/or lymph node-positive cancers involves predicting a presence or absence of lymph node metastasis of gastric cancer. Alternatively, whether a gastric cancer with lymph node metastasis or without metastasis can be determined by the method.

The expression levels of marker genes in a particular specimen can be estimated by quantifying mRNA corresponding to, or protein encoded by, the marker genes. Quantification methods for mRNA are known to those skilled in the art. For example, the levels of mRNAs corresponding to the marker genes can be estimated by Northern blotting or RT-PCR. Since all the nucleotide sequences of the marker genes are known. The GenBank Accession numbers for each marker genes of the present invention are listed in Table 1, Table 2, and FIG. 2. Anyone skilled in the art can design nucleotide sequences of probes or primers to quantify the marker genes.

Also the expression level of the marker genes can be analyzed based on the activity or amount of proteins encoded by the marker genes. A method for determining the amount of marker proteins is shown below. For example, immunoasssays are useful to detect/quantify the protein in a biological material. Any biological material can be used for the detection/quantification of the protein or it's activity. For example, a blood sample is analyzed to determine the protein encoded by serum marker. Alternatively, a suitable method can be selected to determine the activity of proteins encoded by the marker genes according to the activity of each protein analyzed.

Expression levels of the marker genes in a specimen (test sample) are estimated and compared with those in a normal sample. When such a comparison shows that the expression level of a marker gene set forth in Table 1 is higher than that in the normal sample, the subject is judged to be affected with intestinal-type gastric cancer. The expression level of marker genes in specimens from a normal individual and a subject may be determined at the same time. Alternatively, normal ranges of the expression levels can be determined by a statistical method based on the results obtained by analyzing the expression level of the marker genes in specimens previously collected from a control group. A result obtained by examining the sample of a subject is compared with the normal range and when the result does not fall within the normal range, the subject is judged to be affected with intestinal-type gastric cancer.

In the present invention, a diagnostic agent for diagnosing intestinal-type gastric cancer is also provided. The diagnostic agent of the present invention comprises a compound that binds to the DNA or protein of a marker gene. Preferably, an oligonucleotide that hybridizes to the polynucleotide of a marker gene, or an antibody that specifically binds to the protein encoded by a marker gene may be used as the compound. The present invention further provides a method for diagnosing intestinal-type gastric cancer in a subject comprising the step of comparing the marker gene expression profile of a sample specimen collected from a subject with the marker gene expression profile of a control (i.e. a non-cancerous) specimen. When expression profiling analysis shows that the expression profile contains characteristics of intestinal-type gastric cancer, the subject is judged to be affected with the disease. Specifically, when not all but most of the marker genes exhibit intestinal-type gastric cancer-associated patterns of alterations of gene expression levels, the expression profile comprising those of the marker genes has characteristics of intestinal-type gastric cancer. For example, when 50% or more, preferably 60% or more, more preferably 80% or more, still more preferably 90% or more of the marker genes constituting the expression profile exhibit intestinal-type gastric cancer-associated patterns of alterations in gene expression levels, one can safely conclude that the expression profile has characteristics of intestinal-type gastric cancer.

In the diagnostic methods of the present invention, it is preferable that multiple marker genes are selected for comparison of expression levels thereof. The more marker genes selected for comparison, the more reliable the diagnosis. The expression levels of a number of genes can be compared conveniently by using an expression profile. The term “expression profile.” refers to a collection of expression levels of a number of genes, preferably marker genes that are differentially expressed in intestinal type gastric cancers as compared to benign tissues, or differentially expressed between the metastatic and non-metastatic phenotype.

A significant advantage of the inventive methods is that the diagnostic or prognostic determination is made objectively rather than subjectively. Earlier methods were limited because they relied on the subjective examination of histological samples. Another advantage is sensitivity. The methods described herein can discriminate normal, pre-cancerous (i.e., benign adenoma), and cancerous tissue (i.e., gastric carcinoma) very early in the carcinogenic process, whereas subjective histological examination cannot be used for very early detection of pre-cancerous states. The methods also provide valuable information regarding a patients prognosis, i.e., whether the cancer is metastatic or likely to become metastatic.

In a further embodiment, the present invention provides methods for screening candidate agents which are potential targets in the treatment of intestinal-type gastric cancer. As discussed in detail above, by controlling the expression levels or activities of marker genes, one can control the onset and progression of intestinal-type gastric cancer. Thus, candidate agents, which are potential targets in the treatment of intestinal-type gastric cancer, can be identified through screenings that use the expression levels and activities of marker genes as indices. In the context of the present invention, such screening may comprise, for example, the following steps:
(1) contacting a candidate compound with a cell expressing one or more marker genes, wherein the one or more marker genes is selected from the group consisting of the genes listed in Table 1 and Table 2; and
(2) selecting a compound that reduces the expression level of one or more up-regulated marker genes shown in Table 1, as compared to a control or enhances the expression of one or more down-regulated marker genes shown in Table 2 as compared to a control.
   Cells expressing a marker gene include, for example, cell lines established from intestinal carcinoma; such cells can be used for the above screening of the present invention.

Alternatively, the screening method of the present invention may comprise the following steps:
(1) administering a candidate compound to a test animal;
(2) measuring the expression level of one or more marker genes in a biological sample from the test animal, wherein the one or more marker genes is selected from the group consisting of the genes listed in Table 1 and Table 2;
(3) selecting a compound that reduces the expression level of one or more up-regulated marker genes selected from Table 1, as compared to a control or enhances the expression of one or more down-regulated marker genes selected from Table 2, as compared to a control.

Alternatively, the screening method of the present invention may comprise the following steps:
(1) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of the genes listed in Table 1 and Table 2;
(2) measuring the activity of said reporter gene; and
(3) selecting a compound that reduces the expression level of said reporter gene when said marker gene is an up-regulated gene selected from Table 1, or that enhances the expression level of said reporter gene when said marker gene is a down-regulated selected from Table 2, as compared to a control.
   Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

Alternatively, the screening method of the present invention may comprise the following steps:
(1) contacting a candidate compound with a protein encoded by a marker gene, wherein the marker gene is selected from the group consisting of the genes listed in Table 1 and Table 2;
(2) measuring the activity of said protein; and
(3) selecting a compound that reduces the activity of said protein when said marker gene an up-regulated gene selected from Table 1, or that enhances the activity of said protein when said marker gene a down-regulated gene selected from Table 2.
   A protein required for the screening can be obtained as a recombinant protein using the nucleotide sequence of the marker gene. Based on the information of the marker gene, one skilled in the art can select any biological activity of the protein as an index for screening and a measurement method based on the selected biological activity.

In the screening methods of the present invention wherein the expression level of the selected marker gene is decreased in intestinal-type gastric cancer (i.e., down-regulated marker genes), compounds that have the activity to increase, compared to the control, the expression level of the gene should be selected as the candidate agents. Conversely, when a marker gene whose expression level is increased in intestinal-type gastric cancer (i.e., up-regulated marker genes) is selected in the screening method, compounds that have the activity of decreasing the expression level compared to the control should be selected as the candidate agents.

There is no limitation on the type of candidate compound used in the screening of the present invention. The candidate compounds of the present invention can be obtained using any of the numerous approaches of combinatorial library methods known in the art, including: biological library methods; spatially addressable parallel solid phase or solution phase library methods; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233. Libraries of compounds may be presented in solution (e.g., Houghten (1992) Bio Techniques 13:412), or on beads (Lam (1991) Nature 354:82), chips (Fodor (1993) Nature 364:555), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865) or phage (Scott and Smith (1990) Science 249:386; Devlin (1990) Science 249:404; Cwirla et al. (1990) Proc. Natl. Acad. Sci. USA 87:6378; and Felici (1991) J. Mol. Biol. 222:301). (United States Published Patent Application 2002/0103360).

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it. Furthermore, an antibody may be a fragment of an antibody or a modified antibody, so long as it binds to one or more of the proteins encoded by the marker genes. For instance, the antibody fragment may be Fab, F(ab′)², Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

The present invention further provides methods for treating intestinal-type gastric cancer. The present invention revealed that expression levels of certain discriminating marker genes are significantly increased (i.e., up-regulation) or decreased (i.e., down-regulation) in intestinal-type gastric tumors as compared to normal epithelia (see genes listed Tables 1 and 2). Accordingly, any of these marker genes can be used as a target in treating intestinal-type gastric cancer. Specifically, when the expression level of a marker gene is elevated in intestinal-type gastric tumor (up-regulation; e.g., genes of Table 1), then the condition can be treated by reducing expression levels or suppressing its activities. Methods for controlling the expression levels of marker genes are known to those skilled in the art. For example, an antisense nucleic acids or a siRNA corresponding to the nucleotide sequence of the marker gene can be administered to reduce the expression level of the marker gene. Alternatively, an antibody against the protein encoded by the marker gene can be administered to inhibit the biological activity of the protein.

Conversely, when the expression level of a marker gene is decreased in intestinal-type gastric tumors (down regulation; e.g., genes of Table 2), then the condition can be treated by increasing the expression level or enhancing the activity. For example, intestinal-type gastric cancer can be treated by administering a protein encoded by a down-regulated marker gene. The protein may be directly administered to the patient or, alternatively, may be expressed in vivo subsequent to being introduced into the patient, for example, by administering an expression vector or host cell carrying the down-regulated marker gene. Suitable mechanisms for in vivo expression of a gene are known in the art. Alternatively, intestinal-type gastric cancer can be treated by administering an antibody that binds to a protein encoded by an up-regulated marker gene. In a further embodiment, intestinal carcinoma can be treated by administering antisense nucleic acids against an up-regulated marker gene.

In addition to providing methods of treating intestinal-type gastric cancer, the invention also provides methods of preventing intestinal-type gastric cancer, more particularly the onset, progression and metastasis of intestinal-type gastric cancer. Specifically, the present invention provides a method for vaccinating a subject against intestinal-type gastric cancer comprising the step of administering a DNA corresponding to one or more marker genes, proteins encoded by a marker gene, or an antigenic fragment of such a protein, wherein the marker genes comprises a gene up-regulated in intestinal-type gastric cancer, such as those listed in Table 1. The vaccine may comprise multiple vaccine antigens corresponding to multiple up-regulated marker genes.

The present invention provides a method for treating or preventing a cell proliferative disease, such as intestinal-type gastric cancer using an antibody against a polypeptide corresponding to an up-regulated marker gene (e.g., gene of Table 1). According to the method, a pharmaceutically effective amount of an antibody against the polypeptide of the present invention is administered. Since the expression of the genes of Table 1 are up-regulated in intestinal adenocarcinoma cells, and the suppression of the expression of these proteins leads to the decrease in cell proliferating activity, it is expected that intestinal-type gastric cancer can be treated or prevented by binding the antibody and these proteins. Thus, an antibody against a polypeptide encoded by a marker gene of Table 1 is administered at a dosage sufficient to reduce the activity of the corresponding marker protein. Alternatively, an antibody binding to cell surface marker specific for tumor cell can be used as tool for drug delivery. For example, the antibody having a cytotoxic agent are administered at a dosage sufficient to injure the tumor cell.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

The present invention provides preventative and therapeutic vaccines. In the context of the present invention, the term “vaccine” refers to antigenic formulations that induce immunity against intestinal-type gastric tumors. The immunity may be transient and one or more booster administrations may be required.

The antigen within the vaccine may comprise a DNA corresponding to one or more up-regulated marker gene, such as those set forth in Table 1, or a protein encoded by such a marker gene or an antigenic fragment thereof. In the context of the present invention, the term “antigenic fragment” refers to a portion of a molecule, when introduced into the body, stimulates the production of an antibody specific to the marker gene, or induction of cytotoxic lymphocyte against tumors.

The present invention also relates to a method of inducing anti-tumor immunity comprising a step of administering a protein corresponding to an up-regulated marker gene (e.g., gene of Table 1); an immunologically active fragment thereof; or nucleic acids encoding any one of the protein and the fragments thereof. The protein of an up-regulated marker gene of Table 1 or the immunologically active fragment thereof is useful as a vaccine against intestinal-type gastric cancer. In the present invention, vaccine against intestinal-type gastric cancer refers to a substance that has the effect of inducing anti-tumor immunity when it is inoculated upon animals. In general, anti-tumor immunity includes immune responses such as the following:
induction of cytotoxic lymphocytes against tumors,
induction of antibodies that recognize tumors, and
induction of anti-tumor cytokine production.

Therefore, when inoculation of a certain protein into an animal induces any one of these immune responses, the protein is said to have anti-tumor immunity inducing effect. The induction of the anti-tumor immunity by a protein can be detected by observing the response of the immune system in the host against the protein in vivo or in vitro.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting induction of CTL Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

For example, the method of evaluating the inducing action of CTL using dendritic cells (DCs) as APC is well known. DC is a representative APC having the strongest CTL inducing action. In this method, the test polypeptide is initially contacted with DC, and then this DC is contacted with T cells. Detection of T cells having cytotoxic effects against the cells of interest after contacting with DC shows that the test polypeptide has an activity of inducing the cytotoxic T cells. Activity of CTL against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

APC is not limited to DC, and peripheral blood mononuclear cells (PBMCs) may be used. In this case, there are reports that the induction of CTL can be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against intestinal tumor cells are useful as vaccines against intestinal-type gastric cancer. Furthermore, APC that acquired the ability to induce CTL against the intestinal tumors by contacting with the polypeptides are useful as vaccines against intestinal-type gastric cancer. Furthermore, CTL that acquired cytotoxicity due to presentation of the polypeptide antigens by APC can be also used as vaccines against intestinal-type gastric cancer. Such therapeutic methods for treating or preventing intestinal-type gastric cancer using anti-tumor immunity due to APC and CTL are referred to as cellular immunotherapy.

Generally, when using the polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, induction of anti-tumor immunity by a polypeptide can be confirmed by observing the induction of antibody production against the tumor. For example, when antibodies against a polypeptide are induced in a laboratory animal immunized with the polypeptide, and when growth of tumor cells is suppressed by those antibodies, the polypeptide has the ability to induce anti-tumor immunity.

Anti-tumor immunity is induced by administering the vaccine of this invention, and this enables treatment and prevention of intestinal-type gastric cancer. Therapy against cancer, or effect of preventing the onset of cancer may be any one of the following steps, such as inhibitory activity against growth of cancerous cells, involution of cancer, and suppression of occurrence of cancer. Otherwise, it may be decrease of mortality of individuals having cancer, decrease of tumor markers in the blood, alleviation of detectable symptoms accompanying cancer, or such. Such effects are preferably statistically significant, for example, observation, at a significance level of 5% or less, of therapeutic effect against gastric cancer, or preventive effect against cancer onset compared to a control to which the vaccine was not administered is preferred. For example, Student's t-test, the Mann-Whitney U-test, or ANOVA may be used for statistical analyses.

The above-mentioned protein having immunological activity or a vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum, and such, but are not limited thereto. Furthermore, the vaccine of this invention may be combined appropriately with a pharmaceutically acceptable carrier. Examples of such carriers are sterilized water, physiological saline, phosphate buffer, culture fluid, and such. Furthermore, it may contain as necessary, stabilizers, suspensions, preservatives, surfactants, and such. The vaccine is administered systemically or locally. Vaccine administration may be by single administration, or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and after inducing APC or CTL, the cells can be administered to the subject. APC can be also induced by introducing a vector encoding the polypeptide into PBMCs ex vivo. APC or CTL induced in vitro can be cloned prior to administration. By cloning and growing cells which have high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals. Furthermore, a pharmaceutical composition for treating or preventing a cell proliferative disease, such as intestinal-type gastric cancer, comprising a pharmaceutically effective amount of the polypeptide of the present invention is provided. The pharmaceutical composition may be used for raising anti tumor immunity. Thus, polypeptides corresponding to one or more up-regulated marker genes (e.g., gene of Table 1) may be used to treat intestinal-type gastric cancer.

The following examples illustrate aspects of the invention but in no way are intended to limit the scope of the present invention

### EXAMPLES

Prior to the present invention, knowledge of genes involved in intestinal-type gastric tumors was fragmentary. Herein, expression profiles of metastatic and early stage lesions of the intestinal-gastric mucosae were examined and compared to provide information about genes that undergo altered expression during progression to metastasis. The data described herein provides genome-wide information about how expression profiles are altered during multi-step carcinogenesis.

Specifically, to determine genetic mechanisms that underlie development and/or progression of intestinal adenocarcinoma, gene expression profiles of cancer cells obtained by laser-capture microdissection of 20 intestinal-type gastric tumors were compared with expression of genes in corresponding non-cancerous mucosae, using cDNA microarray consisting of 23,040 genes. 62 genes were found to be consistently up-regulated and 76 were consistently down-regulated in cancer tissues tested. Altered expression of 12 of those genes was associated with lymph-node metastasis. A “predictive score,” based on expression profiles of five of the genes that were able to distinguish tumors with metastasis from node-negative tumors in our panel, correctly diagnosed the lymph-node status of four additional gastric cancers. The data provides a valuable index for clinicians to predict metastasis to lymph nodes. The system is also useful to identify novel therapeutic targets for this type of cancer.

Gastric Cancers

Histological studies have classified gastric carcinomas into two distinct groups, namely the intestinal (or differentiated) type and the diffuse (or undifferentiated) type, having different features with regard to epidemiology, etiology, pathogenesis and biological behavior. The intestinal type occurs more commonly in elderly people and has better prognosis, but diffuse-type gastric cancer is seen in relatively younger individuals without preference for either sex and displays a more invasive phenotype with a serious clinical course. Intestinal-type gastric cancer is presumed to result from atrophic gastritis, followed by progression to intestinal metaplasia and/or dysplasia, but the precursor lesion of the diffuse-type tumor is not known.

Epidemiological and experimental studies have revealed that a high intake of smoked, salted and nitrated foods and a low intake of vegetables and fruits increase the risk of gastric cancer and also that Helicobacter pylori infection is a risk factor for the disease. Multiple genetic alterations are involved in gastric tumorigenesis. Loss of heterozygosity (LOH) is observed frequently at loci on chromosomes 1p, 5q, 7p, 12q, 13q, 17p, 18q, and Y. Genetic alterations and/or amplification of oncogenes including K-ras, CTNNB1 (β-catenin), c-erbB-hs 2, K-sam, cycline, and c-met play roles in some gastric cancers, and inactivation of tumor suppressor genes such as p53, RB, APC, DCC and/or CDH1 (E-cadherin) can also be a factor. Germ-line mutation in CDH1 is responsible for disease in a subset of patients with familial gastric cancer, who usually suffer from diffuse-type tumors. Mutations in APC or CTNNB1 are observed preferentially in intestinal-type tumors.

To carry out a comprehensive analysis of altered expression of large numbers of genes in gastric cancer tissues, a genome-wide analysis of gene-expression profiles of intestinal-type gastric cancer tissues was carried out. Tissue samples were obtained by laser-capture microdissection, and RNAs from the tumor cells were hybridized to a cDNA microarray containing 23,040 genes. A set of genes with altered expression in intestinal-type cancers as well as a set associated with lymph node metastasis were defined. The analysis was carried out as follows.

Patients and Tissue Samples

Primary gastric cancers and corresponding non-cancerous gastric mucosae were obtained from 20 patients who underwent gastrectomy. Patient profiles were obtained from medical records. Histopathological classification of each tumor, performed according to the standard Lauren's classification (Lauren et al., 1965, Acta. Path. Microbiol. Scand. 64:31-49) diagnosed all samples as intestinal-type adenocarcinomas. Clinical stage was determined according to the standard UICC TNM classification. The 20 gastric cancer tissues initially analyzed included 18 advanced (T2-T4) and two early (T1) cases. The advanced category included nine node-positive and nine node-negative tumors. No significant differences were seen between node-positive and node-negative patients with respect to age, sex, depth of tumor, or tumor grade. All samples were immediately frozen and embedded in TissueTek OCT medium (Sakura, Tokyo, Japan) and stored at −80° C. until used for microarray analysis.

Laser-Capture Microdissection, Extraction of RNA, and T7-Based RNA Amplification

Cancer cells and non-cancerous gastric epithelium were selectively collected from 5 the preserved samples using laser-capture microdissection. Extraction of total RNA and T7-based amplification were performed using standard methods. 2.5-μg aliquots of amplified RNA (aRNA) from each cancerous and noncancerous tissues were labeled with Cy3-dCTP and Cy5-dCTP, respectively.

cDNA Microarray and Analysis of Data

Fabrication of the cDNA microarray slides, hybridization, washing and detection of signals were carried out using methods known in the art. The fluorescence intensities of Cy5 (non-tumor) and Cy3 (tumor) for each target spot were adjusted so that the mean Cy3/Cy5 ratios of 52 housekeeping genes were equal to one. Because data derived from low signal intensities are less reliable, cut-off values were first determined for signal intensities on each slide and excluded genes for further analysis when both Cy3 and Cy5 dyes gave signal intensities lower than the cut-off. Genes were categorized into three groups according to their expression ratios (Cy3/Cy5): up-regulated (ratio equal to or greater than 2.0), down-regulated (ratio equal to or less than 0.5), and unchanged expression (ratios between 0.5 and 2.0). Genes with Cy3/Cy5 ratios greater than 2.0 or less than 0.5 in more than 75% of the cases examined were defined as commonly up- or down-regulated genes, respectively.

Real-Time Quantitative RT-PCR

Four up-regulated genes (CDH3, NHE1, PLAB, and SOX9) were selected and their expression levels examined by applying the real-time RT-PCR technique (TaqMan PCR, Applied Biosystems, Foster City, Calif.). The Glutaminyl-tRNA synthetase (QARS) gene served as an internal control, because it showed the smallest Cy3/Cy5 fluctuation over experiments. The TaqMan assay was carried out with the same aRNAs used for array analysis, according to the manufacturer's protocol. The PCR reaction was preceded by 95° C. for 10 min, then underwent 40 cycles of 95° C. for 15 s and 60° C. for 1 min. The sequences of primers and probes were as follows:

| | | |
|---|---|---|
| QARS forward primer, | 5′-GGTGGATGCAGCATTAGTG GA-3′ | (SEQ ID NO:1) |
| and | | |
| reverse, | 5′-AAGACGCTCAAA CTGGAACTTGTC-3′; | (SEQ ID NO:2) |
| probe, | 5′-VIC-CTCT GTGGCCCTGGCAAAACCCTT-TAMRA-3′; | (SEQ ID NO:3) |
| CDH3 forward primer, | 5′-CTTCAAAA GTGCAGCCCAGA-3′ | (SEQ ID NO:4) |
| and | | |
| reverse, | 5′-GCAACCTAGGCACACTCAGTATAAAA-3′; | (SEQ ID NO:5) |
| probe, | 5′-FAM-TGGCCGTCCTGCATTT CTGGTTTC-TAMRA-3′; | (SEQ ID NO:6) |
| NME1 forward primer, | 5′-CAGAGAAGGAGATCGGCTTGT G-3′ | (SEQ ID NO:7) |
| and | | |
| reverse, | 5′-CTTGTCATTCAT AGATCCAGTT-3′; | (SEQ ID NO:8) |
| probe, | 5′-FAM-CACCC TGAGGAACTGGTAGATTACACGAGC-TAMRA-3′; | (SEQ ID NO:9) |
| PLAB forward primer, | 5′-GTGC TCATTCAAAAGACCGACA-3′ | (SEQ ID NO:10) |
| and | | |
| reverse, | 5′-GGAAGGACCAGGACTGCTCATA T-3′; | (SEQ ID NO:11) |
| probe, | 5′-FAM-TTAGCCAAA GACTGCCAC-TAMRA-3′; | (SEQ ID NO:12) |
| SOX9 forward primer, | 5′-TGCAAGCATGTGTCATCCA-3′ | (SEQ ID NO:13) |
| and | | |
| reverse, | 5′-AGCAATCCTCAAACTCTCTAGCC-3′; | (SEQ ID NO:14) |
| probe, | 5′-FAM-CTCTGCATCTTCTCTTGGAGTG-TAMRA-3′. | (SEQ ID NO:15) |

Identification of Differentially Regulated Genes and Development of “Prediction Scores”

A random permutation test was carried out to identify “predictor” genes that showed significant differences in mean expression level (Cy3/Cy5) between node-positive and node-negative tumors (Golub et al., 1999, Science 286:531-537). A permutational P value <0.01 was considered to be significant. Subsequently, a forward stepwise discriminant function analysis determined the discriminant coefficient (kj) of a ‘predictor’ gene (j) and constant value (C=−1.945). A “Prediction score (Xi)” was calculated for each sample (i) by the following formula: Xi=Σj kj×log₂(rij)+C

where rij is the expression ratio (Cy3/Cy5) of gene j of sample i. Statistical analyses were performed with the SPSS software package (SPSS Inc., Chicago).

Identification of Commonly Up- or Down-Regulated Genes in Intestinal-Type Gastric Cancers

To determine mechanisms underlying carcinogenesis of the intestinal type of gastric cancer, genes, which were consistently up- or down-regulated in this type of tumor, were identified. A cDNA microarray analysis of more than 20,000 genes in 20 tumors identified 62 genes (including 17 of unknown function) that were up-regulated in more than 75% of the cases examined (Table 1). 76 genes (including 27 of unknown function) were found to be down-regulated in 75% or more of the samples examined (Table 2).

TABLE 1
Genes consistently up-regulated in intestinal gastric cancers

| Symbol | Title | Accession | % up | median | Function |
|---|---|---|---|---|---|
| PROCR | protein C receptor, | L35545 | 100.0 | 3.6 | signal |
| | endothelial (EPCR) | | | | transduction |
| PPI5PIV | phosphatidylinositol (4,5) | U45974 | 100.0 | 4.9 | lipid |
| | bisphosphate 5-phosphatase | | | | metabolism |
| | homolog | | | | |
| NFIL3 | nuclear factor, interleukin 3 | U26173 | 100.0 | 5.0 | transcription |
| | regulated | | | | factor |
| LHX1 | LIM homeobox protein 1 | U14755 | 100.0 | 6.9 | transcription |
| | | | | | factor |
| | EST | H04796 | 100.0 | 12.6 | Unknown |
| | EST | D80822 | 93.3 | 2.6 | Unknown |
| SLC2A1 | Solute carrier family 2 | K03195 | 92.9 | 3.6 | glucose |
| | (facilitated glucose | | | | transport |
| | transporter), member 1 | | | | |
| A | protein. “A” | U47925 | 92.9 | 6.9 | Unknown |
| D6S82E | HLA-B associated | AA234856 | 92.9 | 3.8 | Immune |
| | transcript-5 | | | | |
| CDH3 | cadherin 3, type 1, P- | X63629 | 92.9 | 8.2 | cell adhesion/ |
| | cadherin (placental) | | | | cytoskeleton |
| SLC25A4 | Solute carrier family 25 | J02966 | 92.3 | 2.9 | energy |
| | (adenine nucleotide | | | | generation |
| | translocator), member 4 | | | | |
| PRPS1 | Phosphoribosyl | D00860 | 92.3 | 8.4 | purine base |
| | pyrophosphate synthetase 1 | | | | metabolism |
| MGC5347 | hypothetical protein | AA176698 | 92.3 | 5.3 | Unknown |
| | MGC5347 | | | | |
| GFRA2 | GDNF family receptor | U97145 | 92.3 | 6.7 | cell-cell |
| | alpha 2 | | | | signalling |
| TMEPAI | transmembrane, prostate | AA192445 | 91.7 | 5.2 | Unknown |
| | androgen induced RNA | | | | |
| RPA3 | replication protein A3 | L07493 | 91.7 | 4.9 | DNA repair/ |
| | (14 kD) | | | | Recombination |
| SOX9 | SRY (sex determining | Z46629 | 90.0 | 3.1 | transcription |
| | region Y)-box 9 | | | | factor |
| TFRC | transferrin receptor (p90, | AA806223 | 87.5 | 3.1 | endosome/ |
| | CD71) | | | | receptor |
| HGF | hepatocyte growth factor | M73239 | 87.5 | 4.0 | signal |
| | (hepapoietin A; scatter | | | | transduction/ |
| | factor) | | | | growth factor |
| HSPA9B | heat shock 70 kD protein 9B | L15189 | 86.7 | 3.2 | RNA/protein |
| | (mortalin-2) | | | | processing |
| HRH1 | histamine receptor H1 | D28481 | 85.7 | 3.1 | signal |
| | | | | | transduction |
| DNM1L | dynamin 1-like | AB006965 | 85.7 | 2.7 | mitochondrial |
| | | | | | membrane |
| | | | | | organization |
| | EST | H03296 | 84.6 | 3.0 | Unknown |
| | EST | AI091879 | 84.6 | 3.4 | Unknown |
| TUBA3 | Tubulin, alpha, brain | AA706491 | 83.3 | 3.4 | cell structure |
| | specific | | | | |
| NME1 | non metastatic cells 1, | X17620 | 83.3 | 4.2 | transcription |
| | protein (NM23A) expressed | | | | factor |
| | in | | | | |
| MMP19 | matrix metalloproteinase 19 | U37791 | 83.3 | 5.8 | protein |
| | | | | | degradation |
| LOC51205 | LPAP for lysophosphatidic | AA160670 | 83.3 | 2.9 | lipid |
| | acid phosphatase | | | | metabolism/ |
| | | | | | acid |
| | | | | | phosphatase |
| ENC1 | ectodermal-neural cortex | T03322 | 83.3 | 4.2 | neuronal |
| | (with BTB-like domain) | | | | development |
| CCNC | Cyclin C | M74091 | 83.3 | 3.8 | cell cycle |
| | | | | | control |
| MYBPC2 | myosin binding protein C, | X73113 | 82.4 | 3.2 | Cytoskeletal |
| | fast-type | | | | |
| IRF7 | interferon regulatory factor 7 | U73036 | 82.4 | 3.7 | transcription |
| | | | | | factor |
| HOXB7 | homeo box B7 | M16937 | 82.4 | 4.5 | transcription |
| | | | | | factor |
| RUVBL1 | RuvB (E coli homolog)-like 1 | AB012122 | 81.3 | 2.8 | DNA binding/ |
| | | | | | DNA helicase |
| HSF4 | heat shock transcription | D87673 | 81.3 | 3.1 | transcription |
| | factor 4 | | | | factor |
| | EST | W93907 | 80.0 | 3.5 | Unknown |
| CHST1 | Carbohydrate (keratan | U65637 | 80.0 | 3.8 | polysaccharide |
| | sulfate Gal-6) | | | | metabolism |
| | sulfotransferase 1 | | | | |
| HSPC195 | hypothetical protein | W32401 | 78.9 | 2.4 | Unknown |
| SERPING1 | serine (or cysteine) | M13690 | 78.6 | 8.7 | immune |
| | proteinase inhibitor, clade G | | | | response/ |
| | (C1 inhibitor), member 1 | | | | serine protease |
| | | | | | inhibitor |
| LY6E | lymphocyte antigen 6 | U42376 | 78.6 | 3.5 | signal |
| | complex, locus E | | | | transduction/ |
| | | | | | receptor |
| | EST | AA400550 | 78.6 | 2.1 | Unknown |
| BCL2 | B-cell CLL/lymphoma 2 | M14745 | 78.6 | 6.4 | cell cycle |
| | | | | | regulator/ |
| | | | | | apoptosis |
| | | | | | inhibitor |
| RPL10 | ribosomal protein L10 | AA149846 | 77.8 | 3.4 | protein |
| | | | | | biosynthesis/ |
| | | | | | RNA binding |
| ABCB2 | ATP-binding cassette, sub- | L21204 | 77.8 | 6.0 | peptide |
| | family B (MDR/TAP), | | | | transport |
| | member 2 | | | | |
| SRPX | sushi-repeat-containing | U78093 | 76.9 | 2.2 | Unknown |
| | protein, X chromosome | | | | |
| MIA | melanoma inhibitory | X75450 | 76.9 | 4.3 | cell |
| | activity | | | | proliferation |
| SCD | stearoyl-CoA desaturase | AA452018 | 76.5 | 4.1 | fatty acid |
| | (delta-9-desaturase) | | | | biosynthesis |
| SLC16A2 | Solute carrier family 16 | U05321 | 75.0 | 2.3 | monocarboxylic |
| | (monocarboxylic acid | | | | acid transport |
| | transporters), member 2 | | | | |
| SLC16A1 | Solute carrier family 16 | L31801 | 75.0 | 2.5 | monocarboxylic |
| | (monocarboxylic acid | | | | acid transport |
| | transporters), member 1 | | | | |
| KIAA1247 | similar to glucosamine-6- | AA777773 | 75.0 | 3.4 | Unknown |
| | sulfatases | | | | |
| PRKDC | protein kinase, DNA- | AA670141 | 75.0 | 3.0 | DNA repair/ |
| | activated, catalytic | | | | Recombination |
| | polypeptide | | | | |
| PLAB | prostate differentiation | N30179 | 75.0 | 4.4 | cell-to-cell |
| | factor | | | | signalling |
| PLEK2 | pleckstrin 2 (mouse) | AA308562 | 75.0 | 3.6 | signal |
| | homolog | | | | transduction |
| IFITM2 | interferon induced | X57351 | 75.0 | 3.4 | immune |
| | transmembrane protein 2 | | | | response |
| | (1-8D) | | | | |
| 20D7-FC4 | hypothetical protein | Y10936 | 75.0 | 2.5 | Unknown |
| | Human BAC clone GS1- | AA894447 | 75.0 | 4.2 | Unknown |
| | 99H8 | | | | |
| HRG | histidine-rich glycoprotein | M13149 | 75.0 | 3.5 | blood |
| | | | | | coagulation |
| HSPCB | heat shock 90 kD protein 1, | AI273886 | 75.0 | 2.9 | RNA/protein |
| | beta | | | | processing |
| FHL3 | four and a half LIM | U60116 | 75.0 | 4.3 | muscle |
| | domains 3 | | | | development |
| EIF3S9 | eukaryotic translation | U78525 | 75.0 | 2.4 | protein |
| | initiation factor 3, subunit 9 | | | | synthesis |
| | (eta, 116 kD) | | | | initiation |
| | EST | AA528820 | 75.0 | 2.9 | Unknown |
| CHGB | Chromogranin B) | Y00064 | 75.0 | 4.2 | peptide |
| | (secretogranin 1) | | | | hormone |

Genes whose normalized expression ratio (Tumor/Normal) were >2 in more than 75% of the cases examined were selected. The proportion of up-regulated genes, median values of expression ratios (Cy3/Cy5), and GenBank accession numbers are indicated. Gene functions were summarized from literature sources or according to LocusLink in NCBI (www.ncbi.nlm.nih.gov/LocusLink).

TABLE 2
Genes consistently down-regulated in intestinal gastric cancers

| Symbol | Title | Accession | % down | median | Function |
|---|---|---|---|---|---|
| KHK | ketohexokinase | X78677 | 100.0 | 0.10 | carbohydrate |
| | (fructokinase) | | | | metabolism |
| LOC56287 | CA11 | AI333599 | 100.0 | 0.00 | carbonate |
| | | | | | dehydratase |
| APOA4 | apolipoprotein A-IV | M13654 | 100.0 | 0.01 | lipid |
| | | | | | metabolism |
| ANPEP | alanyl (membrane) | M22324 | 100.0 | 0.05 | Protease |
| | aminopeptidase (CD13, | | | | |
| | p150) | | | | |
| GIF | gastric intrinsic factor | M63154 | 100.0 | 0.00 | small |
| | (vitamin B synthesis) | | | | molecule |
| | | | | | transport |
| RBP2 | retinol-binding protein 2, | AI340234 | 100.0 | 0.02 | vitamin A |
| | cellular | | | | metabolism |
| TFF2 | trefoil factor 2 | AA741431 | 94.4 | 0.13 | defense |
| | (spasmolytic protein 1) | | | | response |
| MAL | mal, T-cell differentiation | M15800 | 93.3 | 0.00 | signal |
| | protein | | | | transduction |
| MTP | microsomal triglyceride | X59657 | 92.9 | 0.02 | lipid |
| | transfer protein (large | | | | metabolism |
| | polypeptide, 88 kD) | | | | |
| | EST | AA788874 | 92.9 | 0.01 | Unknown |
| LOC51237 | hypothetical protein | AA769445 | 92.9 | 0.00 | Unknown |
| APOB | apolipoprotein B | M15421 | 92.3 | 0.00 | lipid |
| | (including Ag(x) antigen) | | | | metabolism |
| MYHL | myosin, heavy | AF127026 | 91.7 | 0.05 | Cytoskeleton |
| | polypeptide-like (110 kD) | | | | |
| GLRX | Glutaredoxin | D21238 | 91.7 | 0.04 | DNA |
| | (thioltransferase) | | | | synthesis/ |
| | | | | | reductase |
| CA2 | carbonic anhydrase II | J03037 | 88.2 | 0.06 | carbonate |
| | | | | | dehydratase |
| IGHM | immunoglobulin heavy | X67292 | 88.2 | 0.03 | Immune |
| | constant mu | | | | |
| ALDH3 | aldehyde dehydrogenase 3 | M77477 | 87.5 | 0.02 | carbohydrate |
| | | | | | metabolism |
| APOA1 | apolipoprotein A-I | J00098 | 87.5 | 0.00 | lipid |
| | | | | | metabolism |
| FBP1 | fructose, 6-bisphosphatase 1 | L10320 | 85.7 | 0.15 | carbohydrate |
| | | | | | metabolism |
| CYP2C9 | cytochrome P450, | M61857 | 85.7 | 0.00 | drug |
| | subfamily IIC | | | | metabolism |
| | (mephenytoin 4- | | | | |
| | hydroxylase), polypeptide 9 | | | | |
| | EST | AI028202 | 85.7 | 0.05 | Unknown |
| TFF1 | trefoil factor 1 | AA614579 | 85.0 | 0.16 | defense |
| | | | | | response |
| | Homo sapiens cDNA: | AA669034 | 85.0 | 0.03 | Unknown |
| | FLJ23125 fis, clone | | | | |
| | LNG08217 | | | | |
| PAXIP1L | PAX transcription | U80735 | 84.6 | 0.06 | transcription |
| | activation domain | | | | factor |
| | interacting protein 1 like | | | | |
| HCF-2 | host cell factor 2 | W37916 | 84.6 | 0.23 | transcription |
| | | | | | factor |
| ATP2A3 | ATPase, Ca++ | Y15724 | 84.2 | 0.21 | small |
| | transporting, ubiquitous | | | | molecule |
| | | | | | transport |
| | | | | | (Ca) |
| FSHPRH1 | FSH primary response | X97249 | 83.3 | 0.14 | spermatogenesis/ |
| | (LRPR1, rat) homolog 1 | | | | oogenesis |
| | EST | AA432388 | 83.3 | 0.03 | Unknown |
| FLJ10846 | hypothetical protein | H06819 | 83.3 | 0.23 | Unknown |
| | FLJ10846 | | | | |
| | EST | AA262280 | 83.3 | 0.06 | Unknown |
| | Homo sapiens | AA573905 | 82.4 | 0.08 | Immune |
| | chromosome 19, cosmid | | | | |
| | R30669 | | | | |
| RNB6 | RNB6 | AI341482 | 82.4 | 0.08 | Unknown |
| IGKC | immunoglobulin kappa | X72475 | 81.3 | 0.07 | Immune |
| | constant | | | | |
| RAB32 | RAB32, member RAS | U59878 | 81.3 | 0.32 | vesicle |
| | oncogene family | | | | transport |
| ADH1 | alcohol dehydrogenase 1 | M12963 | 80.0 | 0.13 | carbohydrate |
| | (class I), alpha polypeptide | | | | metabolism |
| ALDOB | aldolase B, fructose- | X02747 | 80.0 | 0.06 | carbohydrate |
| | bisphosphate | | | | metabolism |
| PAP | pancreatitis-associated | M84337 | 80.0 | 0.01 | cell adhesion/ |
| | protein | | | | proliferation |
| CYP3A7 | cytochrome P450, | D00408 | 80.0 | 0.32 | drug |
| | subfamily IIIA, | | | | metabolism |
| | polypeptide 7 | | | | |
| MT1E | metallothionein 1E | M10942 | 80.0 | 0.03 | heavy metal |
| | (functional) | | | | ion transport |
| MT1H | metallothionein 1H | X64177 | 80.0 | 0.05 | heavy metal |
| | | | | | ion transport |
| | EST | H97976 | 80.0 | 0.16 | Unknown |
| | EST | N58488 | 80.0 | 0.29 | Unknown |
| | EST | AI340056 | 80.0 | 0.10 | Unknown |
| ADH3 | alcohol dehydrogenase 3 | X04299 | 78.9 | 0.09 | carbohydrate |
| | (class I), gamma | | | | metabolism |
| | polypeptide | | | | |
| EEF1E1 | eukaryotic translation | AI290959 | 78.9 | 0.35 | glutathione |
| | elongation factor 1 epsilon 1 | | | | transferase |
| ITIH1 | inter-alpha (globulin) | X16260 | 78.9 | 0.18 | proteinase |
| | inhibitor, H1 polypeptide | | | | inhibitor |
| | EST | AA393089 | 78.9 | 0.33 | Unknown |
| LOC57146 | hypothetical protein from | AF001550 | 78.6 | 0.25 | Unknown |
| | clone 24796 | | | | |
| | EST | T03044 | 78.6 | 0.19 | Unknown |
| | EST | AA719352 | 78.6 | 0.18 | unknown |
| | Homo sapiens mRNA; | W23958 | 78.6 | 0.32 | unknown |
| | cDNA DKFZp434P228 | | | | |
| | (from clone | | | | |
| | DKFZp434P228) | | | | |
| LOC51247 | hypothetical protein | H25172 | 78.6 | 0.11 | unknown |
| | EST | W37871 | 78.6 | 0.13 | unknown |
| CYP3A5 | cytochrome P450, | J04813 | 77.8 | 0.15 | drug |
| | subfamily IIIA | | | | metabolism |
| | (niphedipine oxidase), | | | | |
| | polypeptide 5 | | | | |
| MGAM | maltase-glucoamylase | AF016833 | 76.9 | 0.07 | carbohydrate |
| | (alpha-glucosidase) | | | | metabolism |
| ITGB8 | integrin, beta 8 | AA410685 | 76.9 | 0.24 | cell adhesion/ |
| | | | | | signal |
| | | | | | transduction |
| IREB2 | Iron-responsive element | AA406258 | 76.9 | 0.28 | RNA |
| | binding protein 2 | | | | binding/ |
| | | | | | translational |
| | | | | | regulation |
| PSCA | prostate stem cell antigen | AF043498 | 76.9 | 0.19 | tumor |
| | | | | | antigen |
| PXMP2 | peroxisomal membrane | AI093595 | 76.9 | 0.25 | unknown |
| | protein 2 (22 kD) | | | | |
| LOC63928 | hepatocellular carcinoma | AA527435 | 76.9 | 0.15 | unknown |
| | antigen gene 520 | | | | |
| | EST | AI093836 | 76.5 | 0.32 | unknown |
| LOC51092 | CGI-40 protein | AA458747 | 76.5 | 0.13 | unknown |
| REG1A | Regenerating islet-derived | M18963 | 75.0 | 0.07 | cell |
| | 1 alpha | | | | proliferation |
| INGAP | pancreatic beta cell growth | U41737 | 75.0 | 0.11 | differenciation |
| | factor | | | | |
| SFTPC | surfactant, pulmonary- | N56912 | 75.0 | 0.27 | extracellular |
| | associated protein C | | | | |
| FER1L3 | fer (C. elegans)-like 3 | AI025297 | 75.0 | 0.29 | muscle |
| | myoferlin) | | | | contraction |
| NOS2A | nitric oxide synthase 2A | U31511 | 75.0 | 0.08 | nitric oxide |
| | (inducible, hepatocytes) | | | | synthase |
| RNASE1 | Ribonuclease, RNase A | AA778308 | 75.0 | 0.21 | RNA |
| | family, 1 (pancreatic) | | | | catabolism |
| STAM2 | STAM-like protein | M78581 | 75.0 | 0.30 | signal |
| | containing SH3 and ITAM | | | | transduction |
| | domains 2 | | | | |
| ATP4B | ATPase, H+/K+ | M75110 | 75.0 | 0.16 | small |
| | exchanging, beta | | | | molecule |
| | polypeptide | | | | transport |
| KLF7 | Kruppel-like factor 7 | AI025297 | 75.0 | 0.30 | transcription |
| | (ubiquitous) | | | | factor |
| | EST | H11252 | 75.0 | 0.33 | unknown |
| | DKFZP586A0522 protein | AI306435 | 75.0 | 0.23 | unknown |
| | EST | H23441 | 75.0 | 0.27 | unknown |
| | EST | H79317 | 75.0 | 0.19 | unknown |
| | Homo sapiens clone Hu | L02326 | 75.0 | 0.06 | unknown |
| | lambda7 lambda-like | | | | |
| | protein (IGLL2) gene, | | | | |
| | partial cds | | | | |

Genes whose normalized expression ratio (Tumor/Normal) were <0.5 in more than 75% of the cases examined were selected. The proportion of down-regulated genes, median values of expression ratios (Cy3/Cy5), and GenBank accession numbers are indicated. Gene functions were summarized from literature sources or according to LocusLink in NCBI (www.ncbi.nlm.nih.gov/LocusLink).

Consistently upregulated elements included genes associated with signal-transduction pathways (GFRA2, HGF, HRH1, PLEK2, PLAB, PPI5PIV), genes encoding transcription factors (NFIL3, LHX1, SOX9, IRF7, HOXB7 and HSF4), and genes involved in various metabolic pathways (SCD, CHST1, LPAP, PRPS1), transport systems (TFRC, SLC2A1, SLC16A1, SLC16A2, SLC25A4), cell proliferation (MIA), anti-apotosis (BCL2), protein translation and processing (EIF3S9, HSPA9B, HSPCB, RPL10), DNA replication and recombination (RPA3, RUVBL1, PRDKC), or other functions (NME1, PROCR, SERPING1 and HRG).

Among the consistently down-regulated genes were some that are specific to gastric mucosa and involved in lipid metabolism (MTP, APOB, APOA4, APOA1), carbohydrate metabolism (KHK, ADH3, ALDH3, FBP1, ADH1, ALDOB, MGAM), drug metabolism (CYP2C9, CYP3A7, CYP3A5), carbon dioxide metabolism (LOC56287, CA2), defense response (TFF1, TFF2) or transport of small molecules or heavy metals (ATP2A3, GIF, ATP4B, MT1E, MT1H).

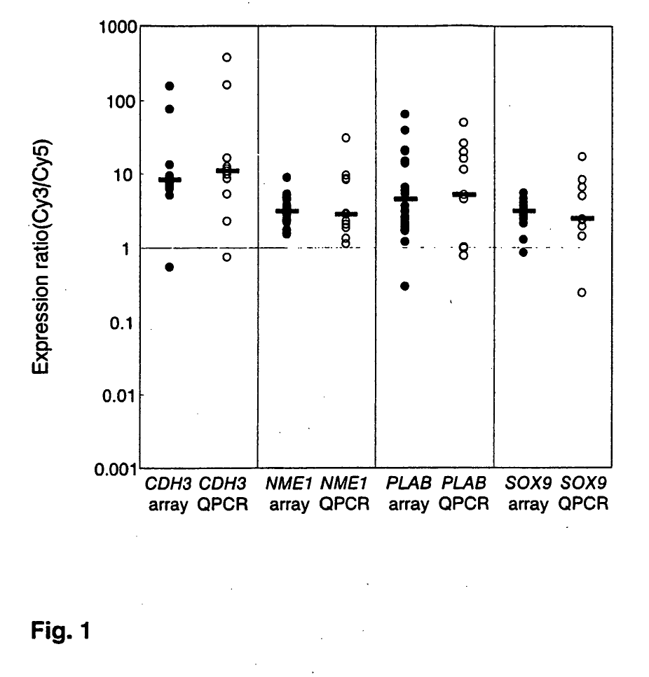

Reproducibility of the data was greater than 85% when genes with signal intensities lower than the cut-off values were excluded. To verify the microarray data further, four commonly up-regulated genes (NMEI, CDH3, PLAB, SOX9) were selected and quantitative RT-PCR was performed using 11 pairs of RNA samples. The results were very similar to microarray data for all four genes (FIG. 1). These data indicate that the analytical approach used was reliable and predictable.

Identification of Genes Associated with Lymph-Node Metastasis

Genes associated with tumor metastasis to lymph nodes were identified. Expression profiles in nine node-positive cases were compared with expression profiles of nine node-negative tumor samples. Twelve genes were identified that were expressed differently (P-value of less than 0.01) by a random-permutation test (FIG. 2A-B). Nine of the 12 genes were relatively up-regulated (DDOST, GNS, NEDD8, LOC51096, CCT3, CCT5, PPP2R1 and two ESTs (GENBANK™ Accession Nos. AAS33633 and AI755112)) and three were down-regulated (UBQLN1, AIM2, USP9X) in node-positive tumors.

Development of “Predictive Scores” for Lymph Node Metastasis

A mathematical equation was developed to achieve a scoring parameter for prediction of lymph node metastasis. Among the 12 genes with statistically significant differences in expression between node-positive and node-negative tumors, a forward stepwise discriminant function analysis identified five as independent “predictors”. The discriminant function analysis examinneds whether an expression level of a gene is varied relate with or without other gene. Five genes that are not influenced to the expression level of other gene have been selected by the analysis. These 5 genes named “predictor”. The “predictive score” was calculated using the expression profiles of these five genes (predictor) and their discriminant coefficients. The “predictive score” has been determined by the following steps;

determining a log expression ratio (Cy3/Cy5) of a gene,

multyplling the discriminant coefficient to the log expression ratio,

summing the values of the discriminant coefficient by log expression ratio (if multiple genes were selected as predictor), and

adding the constant value to the sum,

It was determined that the lymph node metastasis is positive, when the predictive score is plus, or negative when the predictive score is minus.

“Constant value”: the discriminating score, the central value of the average values of each group

When samples are to be classified into two groups, classification of each sample is carried out according to the criterion that to which of the average values of the two groups the value of the sample is closer. Here, a “constant value” can be used for each sample, as a value on the basis of which this analysis is made. Specifically, by setting the discriminating score as 0 (the discriminating score is obtained as the mean (or intermediate) value of the average values of two groups) and determining whether the “constant value” of each sample is positive or negative with respect to the discriminating score, the classification of the samples can be carried out. As a result of the classification, it can be judged whether or not the sample has a disease.

“Discriminant coefficient”: the “weight” of each gene which is involved with the discrimination

“Discriminant coefficient” is obtained by dividing the difference between the average values of two groups by the sum of the standard deviations of the two groups.

The measurement values and the degree of variance thereof are specific for each gene and generally different from those of another gene. Therefore, even when some genes exhibit the same amount of expression, the significance of the “measurement values” thereof varies, depending on the type of the gene (when the degree of variance is high, the significance decreases. Conversely, when the degree of variance is low, the significance increases. In another aspect, the farther the two groups are separated, the larger the significance is. On the contrary, the closer the two groups are, the smaller the significance is). A constant which represents the distance between the two groups when the degree of variance is expressed as 1 is derived from the two values of “variance” and “the distance between the two groups”, which two values are specific to each gene, as described above. This constant is utilized as the “weight” of each gene, when two groups are discriminated from each other.

As shown in FIGS. 2A-B, this scoring system correctly and reliably separated node-positive tumors from node-negative tumors. The robustness of the classification was validated by means of the leave-one-out cross-validation method, i.e., by training on all but one of the samples and using the resulting model to predict the classification for the sample that is left out. Four additional gastric cancer samples were obtained and their “predictive scores” examined. The scores were 1.2, 1.9, −1.0, and −4.3; the former two were independently determined to be positive for node metastasis and the others negative, confirming the reliability of the “predictive score”.

The development of microarray technology has facilitated analysis of expression levels of thousands of genes in a single experiment. This technology is a powerful tool for analyzing genes the expression of which are correlated with pathological phenotypes of various tumors. Based on identification of gene expression patterns, revised classifications of cancer types are made. Gene expression profiles not only have disclosed specific patterns that serve as prognostic markers and drug sensitivity indicators of tumor cells. Genes involved in malignant transformation, progression, and/or metastasis of tumors were identified. The data described herein represents the first genome-wide study of gene expression in microdissected cells from intestinal-type gastric cancers.

Analysis of expression of more than 20,000 genes revealed consistent patterns of expression in intestinal-type gastric cancers. Genes that were commonly altered in the tumors represented fell into several functional categories. Some genes which had been associated with gastric carcinogenesis, such as ERBB2, EGFR and CCNE, were not included in our list because the frequency of their up-regulation in our experiments did not fit the defined criteria for consistently up-regulated genes (i.e., frequency of 75% or more). For example, ERBB2, EGFR and CCNE were reported to be over-expressed in 20%, 50%, and 20% of intestinal gastric cancers respectively, while in the study described herein, those genes showed expression ratios >2) in only 45%, 62.5%, and 10% of the tumors, respectively.

Among the up-regulated genes involved in signal transduction, GFRA2 encodes a glycosyl-phosphatidylinositol-linked cell-surface receptor for neurturin. This receptor forms a complex with the RET transmembrane tyrosine kinase, the over-expression of which is associated with various cancers. The Neurturin/GFRA21RET pathway promotes survival of neurons.

Expression of HGF, the ligand of MET, was also enhanced in the array. The MET proto-oncogene, a receptor-type tyrosine kinase, is involved in cell proliferation and is up-regulated in various other tumors as well. HGF and MET products co-localize in prostate- and breast-cancer cells. The results indicated that over-expression of HGF in gastric-cancer cells activate the HGF/MET signaling pathway in an autocrine manner and play a crucial role in carcinogenesis.

NFLL3, another gene commonly up-regulated in the gastric cancers examined, is regulated by IL-3; its enforced expression in IL-3-deprived cells can prevent apoptosis. This transcription factor regulates a pivotal step in the anti-apoptotic pathway, and its alteration likely contributes to development of human B-cell leukemia. Transcription factor LHX1, which has a unique cysteine-rich zinc-binding domain and is involved in the control of differentiation and development of neural and lymphoid tissues, was also commonly up-regulated on the microarray. Expression of LHX1 has been observed in acute myeloid leukemia cell lines as well as cells from patients with blastic crisis of chronic myeloid leukemia. Expression of HOXB7, a homeobox transcription factor involved in embryonic development, was also frequently elevated in the gastric tumors examined. Altered expression of HOX genes is often involved in leukemias and solid tumors, and over-expression of HOXB7 in immortalized normal ovarian surface epithelium cells dramatically enhances cell proliferation.

Some genes related to intracellular metabolism, DNA replication, and protein synthesis and processing were also up-regulated in our panel of gastric cancers, a result that might reflect accelerated growth and/or cell division. Several genes related to blood coagulation (PROCR, SERPING1 and HRG) were up-regulated as well. PROCR (protein C receptor) binds to protein C, and the complex plays a major role in blood coagulation. PROCR has been detected in several cancer cell lines and its altered expression may explain the complexity of coagulopathy in cancer patients. SERPING1, a C1 inhibitor, has a potentially crucial role in regulating important physiological pathways including complement activation, blood coagulation and fibrinolysis. The HRG product interacts with heparin, thrombospondin and plasminogen. Two effects of HRG protein, inhibition of fibrinolysis and reduction of inhibition of coagulation, indicate a potential prothrombotic effect. Since coagulopathy is common complication among cancer patients, administration of drugs inhibitory to these targets reduce the severity of coagulation defects in patients with gastric cancer.

76 genes, including 27 functionally unknown genes, were down-regulated in more than 75% of the gastric carcinomas examined. This list includes genes involved in metabolism of carbohydrates, lipids and drugs, or in transport of small molecules. Several genes having specific functions in gastric epithelium were down-regulated as well; many of those encode products associated with absorption of nutrients or barriers against bacteria in the intestinal lumen. Down-regulation of these genes reflects “de-differentiation” during carcinogenesis.

Metastasis to lymph nodes is one of the most useful prognostic factors for cancer patients. VEGF C and D play critical roles in this process. However, the complex mechanisms of metastasis cannot be fully explained by alterations in just a few genes. The identification of a set of genes that were differently expressed between node-positive and node-negative tumors provide valuable diagnostic markers and contribute to an improved understanding of the precise biophysical events that lead to metastasis. For example, two of the 12 genes that showed significantly different expression between the two groups are involved in the metabolism of glycoproteins (DDOST, GNS). Glycoproteins are constituents of extracellular matrix (ECM) and cell-surface adhesion molecules. Genes encoding MMPs, uPA, and herapanase are associated with degradation of ECM, a step involved in cancer invasion and metastasis. DDOST and/or GNS mediate a process that modifies proteins associated with cell-adhesion or invasion. In addition, AIM2 (absent in melanoma), a putative tumor suppressor gene, was down-regulated in the node-positive group compared to node-negative tumors.

“Predictive scores” based on expression levels of the five genes (DDOST, GNS, NEDD8, LOC51096, and AIM2), “discriminators”, allow discrimination of node-positive tumors from node-negative tumors with a high probability without the need to remove lymph nodes for examination. This predictive model is a powerful tool for clinical diagnostic and prognostic purposes.

### INDUSTRIAL APPLICABILITY

The gene-expression analysis of intestinal-type gastric cancers described herein, obtained through a combination of laser-capture dissection and genome-wide cDNA microarray, has identified specific genes as targets for cancer prevention and therapy. Based on the expression of a subset of these differentially expressed genes, the present invention provides a method for identifying metastatic intestinal-type gastric tumors. The method of the present invention is a sensitive, reliable and powerful tool that facilitates sensitive, specific and precise diagnosis of such tumors. This system can be specifically utilized in distinguishing malignant from non-malignant tissue as well as early stage cancers from metastatic cancers, particularly those that have undergone lymph node metastasis.

The methods described herein are also useful in the identification of additional molecular targets for prevention, diagnosis and treatment of intestinal-type gastric cancer. The data reported herein add to a comprehensive understanding of gastro-intestinal carcinogenesis, facilitate development of novel diagnostic strategies, and provide clues for identification of molecular targets for therapeutic drugs and preventative agents. Such information contributes to a more profound understanding of gastro-intestinal tumorigenesis, particularly progression to lymph node metastasis, and provide indicators for developing novel strategies for diagnosis, treatment, and ultimately prevention of intestinal adenocarcinoma.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

1. A method of determining whether a tumor is metastatic, comprising comparing the level of expression of a gene in said tumor compared to a control value, wherein said gene is selected from the group consisting of DDOST, GNS, NEDD8, LOC51096, CCT5, CCT3, PPP2R1B and two ESTs (GENBANK™ Accession Nos. AA533633 and AI755112) and wherein an increase in the level of expression in said tumor compared to said control value indicates that the tumor is metastatic.

2. A method of determining whether a tumor is metastatic, comprising comparing the level of expression of a gene in said tumor compared to a control value, wherein said gene is selected from the group consisting of UBQLN1, AIM2, and USP9X and wherein a decrease in the level of expression in said tumor compared to said control value indicates that the tumor is metastatic.

3. The method of claim 1, wherein the expression level is determined by any one methods selected from group consisting of:
(a) detecting the mRNA of the genes
(b) detecting the protein comprising the amino acid sequence encoded by the genes, and
(c) detecting the biological activity of the protein comprising the amino acid sequence encoded by the genes.

4. A method of diagnosing intestinal-type gastric cancer in a subject, the method comprising the steps of:
(a) detecting an expression level of one or more marker genes in a specimen collected from a subject to be diagnosed, wherein the one or more marker genes is selected from the group consisting of the genes listed in Table 1 and the genes listed in Table 2; and
(b) comparing the expression level of the one or more marker genes to that of a control, wherein high expression level of a marker gene from Table 1 or a low expression level of a marker gene from Table 2, as compared to control, is indicative of intestinal-type gastric cancer.

5. A method of predicting lymph node-negative cancers and lymph node-positive cancers, the method comprising the steps of:
(a) detecting an expression level of one or more marker genes in a specimen collected from a subject to be predicted, wherein the one or more marker genes is selected from the group consisting of DDOST, GNS, NEDD8, LOC51096, CCT5, CCT3, PPP2R1B, two ESTs (GENBANK Accession Nos. AA533633 and AI755112), UBQLN1, AIM2, and USP9X; and
(b) comparing the expression level of the one or more marker genes to that of a control, wherein a high expression level or low expression level of a marker gene selected from the group consisting of DDOST, GNS, NEDD8, LOC51096, CCT5, CCT3, PPP2R1B, and two ESTs (GENBANK Accession Nos. AA533633 and AI755112), as compared to the control, is indicative of lymph node-positive cancers or lymph node-negative cancers, respectively, or wherein a low expression level or high expression level of a marker gene selected from the group consisting of UBQLN1, AIM2, and USP9X, as compared to the control, is indicative of lymph node-positive cancers or lymph node-negative cancers.

6. The method of claim 5, wherein the marker gene to be selected is at least one gene selected from the group consisting of DDOST, GNS, NEDD8, LOC51096, and AIM2.

7. The method of claim 6, wherein the marker genes comprise all of DDOST, GNS, NEDD8, LOC51096, and AIM2.

8. The method of claim 7, wherein step (b) further comprises the steps of determining a function of the log ratios of the expression profiles over the selected genes comprising summing the weighted log ratios of the expression profiles over the selected genes, wherein the weight for each gene is a first value when the average log ratio is higher for lymph node-positive cancers than for lymph node-negative cancers and a second value when the average log ratio is lower for lymph node-negative cancers than for lymph node-positive cancers.

9. The method of any one of claim 1-5, wherein the expression level is determined by any one methods selected from group consisting of:
(a) detecting the mRNA of the genes
(b) detecting the protein comprising the amino acid sequence encoded by the genes, and
(c) detecting the biological activity of the protein comprising the amino acid sequence encoded by the genes.

10. The method of claim 9, wherein the expression level of the one or more marker genes is determined by following steps of:
(a) synthesizing aRNA or cDNA of the marker genes from a specimen;
(b) hybridizing the aRNA or cDNA with probes for marker genes; and
(c) detecting the hybridized aRNA or cDNA with the probes quantifying the amount of mRNA thereof.

11. The method of claim 10, wherein the probes are fixed on a DNA array.

12. A method of screening for a therapeutic agent useful in treating or preventing intestinal-type gastric cancer, said method comprising the steps of:
(a) contacting a candidate compound with a cell expressing one or more marker genes, wherein the one or more marker genes is selected from the group consisting of the genes listed in Table 1 and Table 2; and
(b) selecting a compound that reduces the expression level of one or more of the up-regulated marker genes shown in Table 1, as compared to a control, or enhances the expression of one or more of the down-regulated marker genes shown in Table 2, as compared to a control.

13. A method of screening for a therapeutic agent useful in treating intestinal-type gastric cancer, said method comprising the steps of:
(a) administering a candidate compound to a test animal;
(b) measuring the expression level of one or more marker genes in a biological sample from the test animal, wherein the one or more marker genes is selected from the group consisting of the genes listed in Table 1 and Table 2;
(c) selecting a compound that reduces the expression level of one or more of the up-regulated marker genes shown in Table 1, as compared to a control, or enhances the expression of one or more of the down-regulated marker genes shown in Table 2, as compared to a control.

14. A method of screening for a therapeutic agent useful in treating intestinal-type gastric cancer, said method comprising the steps of:
(a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of the genes listed in Table 1 and Table 2;
(b) measuring the activity of said reporter gene; and
(c) selecting a compound that reduces the expression level of said reporter gene when said marker gene is an up-regulated marker gene selected from Table 1, or that enhances the expression level of said reporter gene when said marker gene is a down-regulated marker gene selected from Table 2, as compared to a control.

15. A method of screening for a therapeutic agent useful in treating intestinal-type gastric cancer, said method comprising the steps of:
(a) contacting a candidate compound with a protein encoded by a marker gene, wherein the marker gene is selected from the group consisting of the genes listed in Table 1 and Table 2;
(b) measuring the activity of said protein; and
(c) selecting a compound that reduces the activity of said protein when said marker gene is an up-regulated marker gene selected from Table 1, or that enhances the activity of said protein when said marker gene is a down-regulated marker gene selected from Table 2.

16. The method of any one of claims 12-15, wherein the marker gene is selected from the group consisting of the genes listed in Table 1.

17. The method of any one of claims 12-15, wherein the marker gene is selected from the group consisting of the genes listed in Table 2.

18. A method for treating or preventing intestinal-type gastric cancer, said method comprising the step of administering a compound that is obtained by the method according to any one of claims 12-15.

19. A method for treating or preventing intestinal-type gastric cancer in a subject, said method comprising the step of administering to the subject an antisense nucleic acids or an siRNA against an up-regulated marker gene, wherein said up-regulated marker gene is selected from the group consisting of the genes listed in Table 1.

20. A method for treating or preventing intestinal-type gastric cancer in an subject, said method comprising the step of administering to the subject an antibody or fragment thereof that binds to a protein encoded by an up-regulated marker gene, wherein said up-regulated marker gene is selected from the group consisting of the genes listed in Table 1.

21. A method of treating or preventing intestinal-type gastric cancer in a subject, said method comprising the step of administering to the subject a down-regulated marker gene, or a protein encoded by the gene, wherein said down-regulated marker gene is selected from the group consisting of genes listed in Table 2.

22. A vaccine composition for treating or preventing a intestinal-type gastric tumor, wherein the vaccine composition comprises one or more components selected from the group consisting of:
(a) DNA corresponding to one or more up-regulated marker genes selected from the group consisting of the genes listed in Table 1,
(b) a protein encoded by a DNA of described in (a) above, and
(c) an antigenic fragment of a protein described in (b) above.

23. A method for vaccinating a subject against intestinal-type gastric cancer, the method comprising the step of administering, either alone, or in combination:
(a) a DNA corresponding to one or more up-regulated marker genes selected from the group consisting of the genes listed in Table 1,
(b) a protein encoded by a DNA described in (a) above, or
(c) an antigenic fragment of a protein described in (b) above.

24. The method of claim 2, wherein the expression level is determined by any one methods selected from group consisting of:
(a) detecting the mRNA of the genes
(b) detecting the protein comprising the amino acid sequence encoded by the genes, and
(c) detecting the biological activity of the protein comprising the amino acid sequence encoded by the genes.

25. The method of claim 24, wherein the expression level is determined by any one methods selected from group consisting of:
(a) detecting the mRNA of the genes
(b) detecting the protein comprising the amino acid sequence encoded by the genes, and
(c) detecting the biological activity of the protein comprising the amino acid sequence encoded by the genes.


SEQUENCE LISTING (15 sequences)

SEQ ID NO: 1 - DNA, 21 bp, Artificial Sequence
  Artificially synthesized primer sequence
ggtggatgcagcattagtgga

SEQ ID NO: 2 - DNA, 24 bp, Artificial Sequence
  Artificially synthesized primer sequence
aagacgctcaaactggaacttgtc

SEQ ID NO: 3 - DNA, 24 bp, Artificial Sequence
  Artificially synthesized TaqMan probe sequence
ctctgtggccctggcaaaaccctt

SEQ ID NO: 4 - DNA, 20 bp, Artificial Sequence
  Artificially synthesized primer sequence
cttcaaaagtgcagcccaga

SEQ ID NO: 5 - DNA, 26 bp, Artificial Sequence
  Artificially synthesized primer sequence
gcaacctaggcacactcagtataaaa

SEQ ID NO: 6 - DNA, 24 bp, Artificial Sequence
  Artificially synthesized TaqMan probe sequence
tggccgtcctgcatttctggtttc

SEQ ID NO: 7 - DNA, 22 bp, Artificial Sequence
  Artificially synthesized primer sequence
cagagaaggagatcggcttgtg

SEQ ID NO: 8 - DNA, 22 bp, Artificial Sequence
  Artificially synthesized primer sequence
cttgtcattcatagatccagtt

SEQ ID NO: 9 - DNA, 30 bp, Artificial Sequence
  Artificially synthesized TaqMan probe sequence
caccctgaggaactggtagattacacgagc

SEQ ID NO: 10 - DNA, 22 bp, Artificial Sequence
  Artificially synthesized primer sequence
gtgctcattcaaaagaccgaca

SEQ ID NO: 11 - DNA, 23 bp, Artificial Sequence
  Artificially synthesized primer sequence
ggaaggaccaggactgctcatat

SEQ ID NO: 12 - DNA, 18 bp, Artificial Sequence
  Artificially synthesized TaqMan probe sequence
ttagccaaagactgccac

SEQ ID NO: 13 - DNA, 19 bp, Artificial Sequence
  Artificially synthesized primer sequence
tgcaagcatgtgtcatcca

SEQ ID NO: 14 - DNA, 23 bp, Artificial Sequence
  Artificially synthesized primer sequence
agcaatcctcaaactctctagcc

SEQ ID NO: 15 - DNA, 22 bp, Artificial Sequence
  Artificially synthesized TaqMan probe sequence
ctctgcatcttctcttggagtg